(19) United States
(12) Patent Application Publication
(10) Pub. No.: US 2023/0190113 A1
(43) Pub. Date: Jun. 22, 2023

(54) MATHEMATICAL MODELING OF BLOOD FLOW TO EVALUATE HEMODYNAMIC SIGNIFICANCE OF PERIPHERAL VASCULAR LEGIONS

(71) Applicant: The Regents of the University of California, Oakland, CA (US)

(72) Inventors: Seyed Mehran Mirramezani, Albany, CA (US); Shawn Shadden, Alameda, CA (US)

(21) Appl. No.: 18/000,652

(22) PCT Filed: Jun. 4, 2021
(86) PCT No.: PCT/US2021/035969

§ 371 Date: Dec. 2, 2022

Related U.S. Application Data
(60) Provisional application No. 63/034,801, filed on Jun. 4, 2020.

(51) Int. Cl. A61B 5/02; G16H 50/50

(52) U.S. Cl. CPC A61B 5/02028; G16H 50/50

(57) ABSTRACT

A method for non-invasive assessment of peripheral artery disease (PAD) in the peripheral artery/arteries of a patient can include first constructing from medical image data a source model that includes a patient-specific model of the artery/arteries. The method can further include creating a corresponding benchmark model by replacing stenotic segments with idealized segments in the source model and simulating blood flow and blood pressure in the benchmark model to compute reference hemodynamics information. The method can further include generating an assay model by replacing an idealized artery/arteries of interest in the benchmark model with the actual stenotic geometry of the artery/arteries from the source model.

### CROSS-REFERENCE TO RELATED APPLICATION

The present application claims priority to U.S. Provisional Application No. 63/034,801 entitled “NON-INVASIVE TECHNIQUE FOR ASSESSMENT OF PERIPHERAL ARTERY DISEASE”, and filed on Jun. 4, 2020. The entire contents of the above-listed application is hereby incorporated by reference for all purposes.

### FIELD

The present description relates generally to peripheral computed tomographic angiography (pCTA), and more particularly to the performance of pCTA-based blood flow modeling in diagnosing functionally significant peripheral lesions.

### BACKGROUND

The prevalence of peripheral vascular disease (PVD) continues to increase, is a major cause of morbidity and mortality and poses a significant financial burden on health care systems. Treatment options are based upon the presence and severity of symptoms, evidence of rest pain and/or non-healing ulcers or gangrene and clinical tests to evaluate disease severity. Revascularization, either with surgical bypass or peripheral vascular intervention (PVI), reduces symptoms in those with intermittent claudication and improves wound healing in those with critical limb ischemia if applied to flow limiting lesions.

Evaluating the severity of lesions in peripheral arteries can be challenging due to lesion eccentricity, adjacent vessel calcification and imaging artifacts. Conventional imaging techniques include arterial duplex ultrasound, computed tomographic angiography (CTA), and digital subtraction angiography (DSA). These methods are useful for anatomic assessment but cannot determine the functional significance of lesions. Invasive measurement of trans-lesion pressure drop can provide functional hemodynamic information, but it is under-utilized due to the risk, complexity, and cost.

Physiologic lesion assessment in the coronary vasculature is well validated and widely accepted into routine clinical practice. The traditional approach requires the use of a 0.014-inch wire that is placed distal to the lesion, measurement of a resting drop and then use of medications to induce maximal hyperemia. A FFR value of 0.8 or less identifies a coronary lesion with a high likelihood of causing ischemia. This methodology is based upon a pressure-derived index of the maximal achievable myocardial blood flow in the presence of an epicardial stenosis. More recently, resting pressure indices using the instantaneous wave-free ratio (iFR) have been developed. Rotational angiography alone, without the use of a pressure wire or medications to induce maximal hyperemia, have also been developed and are referred to as virtual FFR. More relevant to the current study is the use of CT data sets to estimate FFR, referred to as CT-derived FFR, FFR_{CT}.

The ability to accurately assess the functional significance of peripheral vascular lesions can have relevance for pre-procedural planning. Surgical bypass or PVI could then be targeted to only those lesions that are hemodynamically significant. Currently, there are no standardized methods or techniques to assess the functional significance of lesions located in the peripheral vasculature. The measurement of pressure drop using catheters and/or wires placed into the peripheral vasculature is cumbersome and time-consuming and therefore, is not routinely performed.

As such, there remains a need for improved techniques for assessing a patient's peripheral artery disease.

### SUMMARY

Certain implementations of the disclosed technology include image-based blood flow modeling from peripheral computed tomographic angiography (pCTA) which may provide means for non-invasive methods and systems configured to determine the hemodynamic significance of a patient's lesions.

Certain implementations may be used to non-invasively evaluate a patient's PAD by determining which patients require further (e.g., invasive) diagnostic testing or treatment. Alternatively or in addition thereto, such implementations may include determining which vascular segments in a patient require interventional treatment. Alternatively or in addition thereto, such implementations may include predicting the functional improvement in a patient's blood flow by interventional treatment of an artery or arteries, which can be advantageously used as potential effective endpoints in clinical practice.

It should be understood that the brief description above is provided to introduce in a simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a flow diagram illustrating an example of a method that includes steps for image-based modeling of blood flow in a representative patient, according to an embodiment;

FIG. 2 illustrates multiple representative modeling results from the method illustrated by FIG. 1; and

FIG. 3 shows a flowchart illustrating a method for non-invasive assessment of peripheral artery disease (PAD) in at least one peripheral artery of a patient.

### DETAILED DESCRIPTION

Non-invasive systems and methods for evaluation of the hemodynamic significance of lesions can potentially improve diagnosis and treatment planning in patients with PVD. Image-based computational modeling of blood flow has shown to be a powerful tool to extract relevant hemodynamic information from non-invasive medical image data such as CTA. For example, the FFR_{CT} method has recently emerged as the de facto standard to non-invasively compute coronary fractional flow reserve (FFR) and has demonstrated high diagnostic performance against invasive FFR measurement for identification of patients with coronary lesions causing ischemia.

Certain implementations may include systems and/or methods for an image-based modeling procedure in which a vascular model may be constructed by segmenting the peripheral arteries from a CTA image volume. Computational fluid dynamics may then be used to compute flow and pressure throughout the model. Implementations may include a novel, non-invasive method for evaluating the functional significance of lesions within the peripheral vasculature having high diagnostic accuracy using both resting and exercise pressure drop.

A pilot study of the disclosed technology included evaluating the sensitivity, specificity and accuracy of a non-invasive method based on image-based computational fluid dynamic (CFD) simulation, integrating anatomical, physiological and hemodynamic information, as a novel tool to determine if peripheral arteries lesions are hemodynamically significant and to estimate the number of patients needed for a properly sized validation study. The diagnostic performance of this method was evaluated against visual assessment of invasive DSA imaging.

In the study, 212 patients undergoing lower extremity DSA for clinical indications were identified from the Cardiac Catheterization Laboratory database. All patients had critical limb ischemia or intermittent claudication and were referred for peripheral CTA and DSA as part of their routine clinical care. Peripheral CTA was done prior to elective DSA in all patients. Only patients having peripheral CTA within 20 days of the DSA were included, yielding 10 patients. This retrospective study was approved by waiver of consent by the Institutional Review Board at USC. Independent research personnel blinded to the objectives of the study obtained all the pre-specified demographics and variables, Variables were entered into a dedicated study database for analysis.

For both DSA and peripheral CTA data sets, the peripheral arteries were divided into 8 segments per extremity (i.e., common iliac, external iliac, common femoral, superficial femoral, popliteal, anterior tibial, posterior tibial and peroneal arteries). For DSA data sets, stenosis severity was graded by visual estimation as: normal (grade 0), mildly stenotic (grade I), moderately stenotic (grade II), severely stenotic (grade III) or occluded (grade IV). If a segment could not be appropriately visualized, it was not analyzed for stenosis severity and was classified as non-evaluable. An investigator (MM) blinded to the results of DSA calculated a resting pressure drop (RPD) and exercise pressure drop (ExPD) for each segment from image-based blood flow modeling described below.

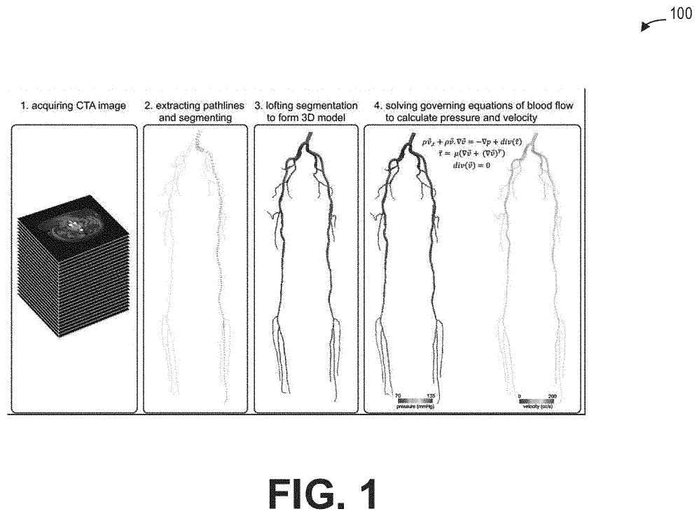

FIG. 1 shows a diagram illustrating an example of a method 100 that includes steps for image-based modeling of blood flow in a representative patient. An image-based computer model of the peripheral arteries can be constructed by creating paths along each vascular segment and segmenting the lumen along each path, thus resulting in 3D computer model. Blood flow and pressure may be modeled according to a reduced order modeling procedure, which requires specification of the flow rate at the model inlet and resistance boundary conditions at the model outlets.

The inflow rate was estimated as a fraction of each patient's cardiac output (Qᵢₙ=α×cardiac output, α<1). The cardiac output was approximated form a recent study (13): cardiac output=2.4×body surface area. The value of a was defined based on in vivo flow measurements. Namely, it was assumed that roughly ⅔ of cardiac output reaches the supraceliac aorta, from which approximately 30% goes to the infrarenal aorta, resulting in an α≈0.2.

Resistance values were set so that computed mean blood pressure at the inlet matched clinical target values. To simulate exercise, it was assumed that cardiac output increased due to change in heart rate and stroke volume (i.e., cardiac output=stroke volume×heart rate). Exercise heart rate was approximated by HR_{ex.}=0.85 (220−age), and the left ventricular stroke volume index (ml/m²) changed based on age. The increase in diastolic and systolic pressure due to exercise was approximated based on age and gender for each patient.

The computed pressure fields enabled assessment of pressure reduction through each artery or arterial segment. In order to isolate the effect of disease, the system differentiated how much pressure reduction occurred due to disease versus ordinary pressure reduction occurring in the absence of disease. This was accomplished by first constructing a 3D computer model of the arteries from the image data. This model was considered the “source model.” A corresponding “benchmark model” was created by replacing stenotic (diseased) segments with idealized segments. Blood flow and pressure were simulated in the benchmark model using the methods described above to compute reference pressure drop. An “assay model” was then generated by replacing an idealized artery (or arteries) of interest in the benchmark model with the actual stenotic geometry of the artery (or arteries) from the source model, and then re-computing pressure drop.

Reference pressure drop calculated from the benchmark model were differenced from the pressure drop computed from the assay model. This was performed for both rest and exercise conditions. A cutoff value for the deviation in RPD and ExPD between the benchmark and assay models was used to classify the functional significance of a diseased artery (or arteries). An in-house Python framework or other suitable framework may be developed to automate the computational procedure described above.

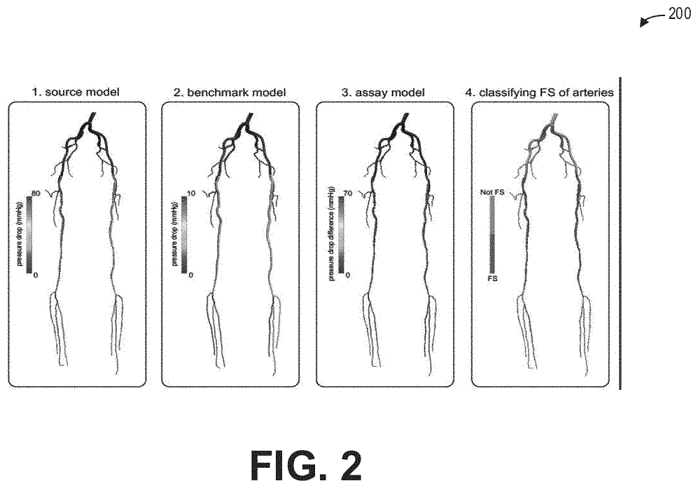

FIG. 2 illustrates multiple representative modeling results 200 from the method 100 illustrated by FIG. 1. Pressure drop are computed in a source model representing the in-vivo conditions (left). Pressure drop are computed in benchmark model in which all stenotic segments are virtually corrected (middle left). Pressure drop computed in an assay model (middle right), which re-introduces disease segment(s) of interest to the benchmark model. By comparing the pressure drop between the assay model and benchmark model, the functional significance of the diseased segment(s) of interest can be evaluated.

A functionally significant (FS) lesion was defined as grade III or IV by DSA. From peripheral image-based blood flow modeling, a FS lesion was defined as an RPD>5 mmHg. Analysis was repeated defining a FS lesion as an ExPD>20 mmHg. Categorical variables are presented as number and percentage. Continuous variables are presented as mean±standard deviation. Sensitivity, specificity and accuracy were determined using an RPG>5 mmHg and an ExPD>20 mmHg with DSA as the gold standard.

In the case study, ten patients with symptomatic PVD were evaluated with both peripheral CTA and DSA (see Table 1 below). The mean age was 52±16 years, 4 (40%) were male, 8 (80%) presented with critical limb ischemia and 2 (20%) presented with intermittent claudication. There was a high prevalence of diabetes mellitus (50%), hypertension (60%) and chronic kidney disease (50%). The mean ankle brachial index was 0.60±0.29. Sixty-six segments were evaluable by both imaging methods.

TABLE 1
Patient Demographics

| | |
|---|---|
| Age, years, mean ± SD | 52 ± 16 |
| Gender | |
| Men | 4 (40%) |
| Women | 6 (60%) |
| Critical limb ischemia | 8 (80%) |
| Hypertension | 6 (60%) |
| Hyperlipidemia | 2 (20%) |
| Diabetes mellitus | 5 (50%) |
| Tobacco use | 1 (10%) |
| Coronary artery disease | 1 (10%) |
| Congestive heart failure | 1 (10%) |
| Chronic kidney disease | 5 (50%) |
| Ankle brachial index, mean ± SD | 0.60 ± 0.29 |

Twenty segments (30%) were classified as FS and forty-six segments (70%) were classified as not FS by DSA assessment. Using an RPG>5 mmHg, sensitivity was 80%, specificity was 85% and accuracy was 79% (see Table 2 below). Using an ExPD>20 mm Hg, sensitivity was 84%, specificity was 89% and accuracy was 88%.

TABLE 2
Sensitivity, specificity and accuracy of blood
flow modeling of peripheral computed tomography
compared to digital subtraction angiography

| | Sensitivity | Specificity | Accuracy |
|---|---|---|---|
| | (%) | (%) | (%) |
| Resting pressure drop > 5 mm Hg | 80 | 85 | 79 |
| Exercise pressure drop > 20 mm Hg | 84 | 89 | 88 |

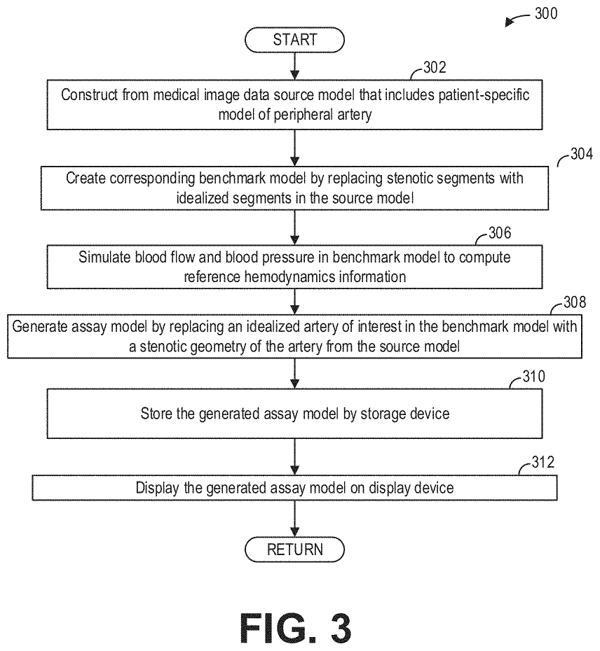

FIG. 3 shows a flowchart illustrating a method 300 for non-invasive assessment of peripheral artery disease (PAD) in at least one peripheral artery of a patient.

At 302, a source model that includes a patient-specific model of the at least one peripheral artery is constructed from medical image data.

At 304, a corresponding benchmark model is created by replacing stenotic segments with idealized segments in the source model.

At 306, blood flow and blood pressure are simulated in the benchmark model to compute reference hemodynamics information.

At 308, an assay model is generated by replacing at least one idealized artery of interest in the benchmark model with an actual stenotic geometry of the at least one artery from the source model.

In an optional step at 310, the generated assay model may be stored by a storage device such as a memory.

In an optional step at 312, the generated assay model may be displayed by a display device such as a monitor.

Aspects of the disclosure may operate on particularly created hardware, firmware, digital signal processors, or on a specially programmed computer including a processor operating according to programmed instructions. The terms controller or processor as used herein are intended to include microprocessors, microcomputers, Application Specific Integrated Circuits (ASICs), and dedicated hardware controllers.

One or more aspects of the disclosure may be embodied in computer-usable data and computer-executable instructions, such as in one or more program modules, executed by one or more computers (including monitoring modules), or other devices. Generally, program modules include routines, programs, objects, components, data structures, and so on, that perform particular tasks or implement particular abstract data types when executed by a processor in a computer or other device. The computer executable instructions may be stored on a computer readable storage medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGAs, and the like.

Particular data structures may be used to more effectively implement one or more aspects of the disclosure, and such data structures are contemplated within the scope of computer executable instructions and computer-usable data described herein.

The disclosed aspects may be implemented, in some cases, in hardware, firmware, software, or any combination thereof. The disclosed aspects may also be implemented as instructions carried by or stored on one or more or computer-readable storage media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media.

Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Communication media means any media that can be used for the communication of computer-readable information. By way of example, and not limitation, communication media may include coaxial cables, fiber-optic cables, air, or any other media suitable for the communication of electrical, optical, Radio Frequency (RF), infrared, acoustic or other types of signals.

An example can include a method for non-invasive assessment of peripheral artery disease (PAD) in at least one peripheral artery of a patient, the method comprising: constructing from medical image data a source model that includes a patient-specific model of the at least one peripheral artery; creating a corresponding benchmark model by replacing stenotic segments with idealized segments in the source model; simulating blood flow and blood pressure in the benchmark model to compute reference hemodynamics information; and generating an assay model by replacing at least one idealized artery of interest in the benchmark model with an actual stenotic geometry of the at least one artery from the source model. One or more tangible, non-transitory computer-readable media storing executable instructions that, when executed by a processor, cause the processor to perform the method.

An example can include a system for non-invasive assessment of peripheral artery disease (PAD) in at least one peripheral artery of a patient, the system including a processor configured to: construct from medical image data a source model that includes a patient-specific model of the at least one peripheral artery; create a corresponding benchmark model by replacing stenotic segments with idealized segments in the source model; simulate blood flow and blood pressure in the benchmark model to compute reference hemodynamics information; and generate an assay model by replacing at least one idealized artery of interest in the benchmark model with an actual stenotic geometry of the at least one artery from the source model.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for non-invasive assessment of peripheral artery disease (PAD) in at least one peripheral artery of a patient, the method comprising:
constructing from medical image data a source model that includes a patient-specific model of the at least one peripheral artery;
creating a corresponding benchmark model by replacing stenotic segments with idealized segments in the source model;
simulating blood flow and blood pressure in the benchmark model to compute reference hemodynamics information; and
generating an assay model by replacing at least one idealized artery of interest in the benchmark model with an actual stenotic geometry of the at least one artery from the source model.

2. The method of claim 1, wherein the stenotic segments correspond to disease.

3. The method of claim 1, wherein the hemodynamics information includes information pertaining to a drop in the patient's blood pressure.

4. The method of claim 1, further comprising using a cutoff value for a deviation in at least one hemodynamic parameter to classify a functional significance of a diseased artery.

5. The method of claim 4, wherein the at least one hemodynamic parameter includes a drop in the patient's blood pressure.

6. The method of claim 1, further comprising a storage device storing the generated assay model.

7. One or more tangible, non-transitory computer-readable media storing executable instructions that, when executed by a processor, cause the processor to perform the method of claim 1.

8. A system for non-invasive assessment of peripheral artery disease (PAD) in at least one peripheral artery of a patient, the system including a processor configured to:
construct from medical image data a source model that includes a patient-specific model of the at least one peripheral artery;
create a corresponding benchmark model by replacing stenotic segments with idealized segments in the source model;
simulate blood flow and blood pressure in the benchmark model to compute reference hemodynamics information; and
generate an assay model by replacing at least one idealized artery of interest in the benchmark model with an actual stenotic geometry of the at least one artery from the source model.

9. The system of claim 8, wherein the stenotic segments correspond to disease.

10. The system of claim 8, wherein the hemodynamics information includes information pertaining to a drop in the patient's blood pressure.

11. The system of claim 8, wherein the processor is further configured to use a cutoff value for a deviation in at least one hemodynamic parameter to classify a functional significance of a diseased artery.

12. The system of claim 11, wherein the at least one hemodynamic parameter includes a drop in the patient's blood pressure.
